(19) United States
(12) Patent Application Publication
(10) Pub. No.: US 2021/0380951 A1
(43) Pub. Date: Dec. 9, 2021

(54) RNA-BASED METHODS TO LAUNCH HEPATITIS B VIRUS INFECTION

(71) Applicant: The Rockefeller University, New York, NY (US)

(72) Inventors: Charles Rice, New York, NY (US); Yingpu Yu, New York, NY (US); William Schneider, New York, NY (US)

(21) Appl. No.: 17/281,760

(22) PCT Filed: Sep. 19, 2019
(86) PCT No.: PCT/US19/51909

§ 371 Date: Mar. 31, 2021

Related U.S. Application Data
(60) Provisional application No. 62/741,032, filed on Oct. 4, 2018.

(51) Int. Cl. C12N 7/00; C12Q 1/70; A01K 67/027

(52) U.S. Cl. CPC C12N 7/00; C12Q 1/706; A01K 67/0271; A01K 2267/0337; C12Q 2600/156; A01K 2207/12; A01K 2227/105; C12N 2730/10151

(57) ABSTRACT

This disclosure describes a method to induce HBV infection in cells or animal models with an HBV pregenomic RNA (pgRNA). The method is amenable to multiple genotypes and has excellent signal-to-noise ratios. The method can be used to identify novel anti-HBV agents, measure anti-HBV drug efficiency, and predict drug resistance.

### CROSS-REFERENCE TO RELATED APPLICATIONS

This application claims priority under 35 U.S.C. § 119(e) to U.S. Provisional Patent Application No. 62/741,032, filed Oct. 4, 2018. The foregoing applications are incorporated by reference herein.

### FIELD OF THE INVENTION

This disclosure relates to methods for initiating hepatitis B virus (HBV) infection and more specifically relates to methods for initiating HBV infection in vitro and in vivo with HBV pregenomic RNA (pgRNA).

### BACKGROUND OF THE INVENTION

HBV is a small DNA virus, transmitted by blood, which exclusively infects human hepatocytes. Upon HBV entry into hepatocytes, its relaxed circular, partially double-stranded DNA (dsDNA) genome is converted in the nucleus to the stable episomal form known as covalently closed circular DNA (cccDNA). cccDNA is transcribed by host RNA polymerase II (Pol II) to produce all HBV RNAs, and cccDNA persistence in hepatocytes is thought to be responsible for chronicity.

HBV chronically infects over 250 million people worldwide and claims an estimated 880,000 lives each year. There is a vaccine to prevent infection, but no effective cure for chronic infections. For chronically infected patients, there are two main therapies: orally administered nucleoside analogs and injectable interferon alpha (IFNα)—a naturally occurring cytokine that elicits a broad antiviral response. Nucleoside analogs suppress HBV replication but rarely eliminate the virus. In contrast, IFNα can cure chronic HBV, but the success rate is only ˜10%.

The cure rate for chronic HBV has stagnated for decades, but recently, there have been renewed efforts to develop therapies to cure chronic HBV. However, a major challenge for HBV research and drug development is that signal-to-noise ratios for many assays that detect HBV proteins and nucleic acids are poor, and it is difficult to predict drug-resistant viral variants prior to testing in the clinic.

Accordingly, there remains a pressing need for methods to induce HBV infection in cells that overcome these challenges.

### SUMMARY OF THE INVENTION

This disclosure describes a method to launch HBV infection with an HBV pregenomic RNA (pgRNA) that overcomes these challenges. The method is amenable to multiple genotypes and has excellent signal-to-noise ratios. The method can be used to identify novel anti-HBV agents, measure anti-HBV drug efficiency, and predict drug resistance.

In one aspect, the method includes contacting a cell with a nucleic acid molecule comprising an HBV pgRNA from an HBV genotype and culturing the cell under a condition to initiate reverse transcription from the HBV pregenomic RNA and HBV replication. The method may further include determining a level of HBV infection by quantifying the amount of one or more HBV proteins or HBV nucleic acids. The HBV proteins may include HBV surface antigens, such as HBsAg. Quantification of the amount of the HBV proteins may be performed by CLIA, and quantification of the amount of the HBV nucleic acids may be performed by qPCR.

In some embodiments, the nucleic acid molecule may be in vitro transcribed from an HBV DNA of the HBV genotype. The nucleic acid molecule may be a 3′-polyadenylated and/or 5′-capped nucleic acid molecule. In some embodiments, the cell may be a human cell or a higher primate cell. The cell may be a HepG2 cell, a HepG2-NTCP cell, a Huh 7 cell, a Huh-7.5 cell, a Huh7.5-NTCP cell, a HepRG cell, a 293T cell, an HLC cell, or a human primary hepatocyte (PHH). In some embodiments, the HBV genotype is selected from the group consisting of HBV genotypes A, B, C, D, E, F, G, H, I, and J.

In a second aspect, this disclosure also provides a method for screening an HBV modulator for the ability to increase or decrease HBV infection. The method includes: (a) providing a cell having a nucleic acid molecule comprising an HBV pgRNA from an HBV genotype; (b) determining an amount of one or more HBV proteins or HBV nucleic acids in the presence of an HBV modulator; and (c) determining whether the HBV modulator increases or decreases HBV infection by comparing the amount to a reference amount that is obtained in the same manner except in the absence of the HBV modulator. The HBV modulator increases HBV infection if the amount is higher than the reference amount, and the HBV modulator decreases HBV infection if the amount is lower than the reference amount.

In some embodiments, the HBV modulator is an anti-HBV modulator, having the ability to decrease HBV infection. Examples of the HBV modulator may include a small molecule, an antibody, an aptamer, or an inhibitory nucleic acid molecule.

In a third aspect, this disclosure also provides a method of identifying an HBV viral variant with resistance or decreased sensitivity to an HBV modulator. The method includes (a) contacting a cell with a nucleic acid molecule comprising an HBV pgRNA from an HBV genotype; (b) contacting the cell with an HBV modulator; (c) culturing the cell to form a cell culture under a condition to initiate reverse transcription from the HBV pregenomic RNA and HBV replication; (d) obtaining an HBV DNA from the cell or a supernatant in the cell culture; and (e) determining a mutation in the nucleotide sequence of the HBV DNA by sequencing the obtained HBV DNA. An HBV viral variant having the mutation is identified as the HBV viral variant with resistance or decreased sensitivity to the HBV modulator. In some embodiments, sequencing the obtained HBV DNA is carried out by next-generation sequencing (NGS).

The present disclosure also provides a method for enriching mutations of an HBV viral variant. The method may include: (f) obtaining a second nucleic acid molecule comprising an HBV pgRNA by in vitro transcription from the obtained HBV DNA and repeating steps (a) to (d), (f), and optionally step (e) one or more times.

In a fourth aspect, this disclosure also provides a method of introducing HBV infection in an animal model. The method includes introducing into the liver of an animal model a nucleic acid molecule comprising an HBV pgRNA from an HBV genotype. In some embodiments, the method may include introducing into the larger liver lobe of the animal model a nucleic acid molecule comprising an HBV pgRNA from an HBV genotype. In some embodiments, the animal model is a human liver chimeric mouse. The human liver chimeric mouse may have a high degree of human hepatocyte engraftment.

In a fifth aspect, this disclosure also provides a kit for introducing an HBV genome in a cell. The kit includes a DNA molecule comprising a binding site for an RNA polymerase and a DNA sequence encoding an HBV pgRNA from an HBV genotype. In some embodiments, the kit may include a plurality of DNA molecules generated by randomizing individual codons, each of the plurality of DNA molecules having a binding site for an RNA polymerase and a DNA sequence encoding an HBV pregenomic RNA from an HBV genotype. In some embodiments, the kit further includes an RNA polymerase, optionally one or more nucleoside triphosphates, and a buffer. This disclosure further provides a kit for introducing an HBV genome in a cell. The kit includes a nucleic acid molecule comprising an HBV pgRNA from an HBV genotype. The nucleic acid molecule may be in vitro transcribed from an HBV DNA of the HBV genotype. In some embodiments, the nucleic acid molecule may be a 3′-polyadenylated and/or 5′-capped nucleic acid molecule. In some embodiments, the HBV genotype is selected from the group consisting of HBV genotypes A, B, C, D, E, F, G, H, I, and J.

The foregoing and other objects, features, and advantages of the present disclosure set forth herein will be apparent from the following description of particular embodiments of those inventive concepts, as illustrated in the accompanying drawings. The drawings depict only typical embodiments of the present disclosure and, therefore, are not to be considered limiting in scope.

### BRIEF DESCRIPTION OF THE DRAWINGS

FIGS. 1A, 1B, 1C, and 1D are a set of diagrams and photographs showing that in vitro-transcribed HBV pgRNA initiates virus replication and forms cccDNA in Huh-7.5-NTCP cells. FIG. 1A shows schematic of HBV pgRNA transfection. HBV pgRNA transfection initiates HBV replication in cell culture. Transfecting in vitro-transcribed HBV pgRNA bypasses the early stages of infection. Transfected pgRNA is translated to produce core and Pol proteins, which together package and reverse-transcribe pgRNA within intracellular particles. The resulting relaxed circular DNA (rcDNA) recycles to the nucleus where it is repaired by host enzymes to form cccDNA, the template for all HBV mRNAs. cccDNA transcription by host Pol II produces additional HBV mRNAs, including that which encodes HBsAg. FIG. 1B shows qPCR of intracellular HBV DNA. WT stands for wild-type; YMHD indicates an HBV polymerase defective mutant. The dotted line indicates a limit of quantification. FIG. 1C shows CLIA quantification of HBsAg in transfected cell supernatants. IU stands for international units. N.D. stands for not detected. FIG. 1D shows Southern blot hybridization of Hirt-extracted HBV DNA with [α-32P] dATP-labeled probe. 85° C. denatures relaxed circular DNA to single-stranded DNA, leaving supercoiled cccDNA intact. Digestion with EcoRI linearizes cccDNA and shifts the band up. RC/L stands for relaxed circular/linear; CCC stands for covalently closed circular; SS stands for single-stranded DNA; kb stands for kilobases.

FIGS. 2A, 2B, 2C, 2D, and 2E are a set of diagrams and photographs showing that injecting HBV pgRNA into huFNRG mouse liver initiates HBV infection. FIG. 2A shows qPCR of HBV DNA in mouse serum after pgRNA injection. GE stands for genome equivalents. LOQ stands for a limit of quantification. FIG. 2B shows qPCR of HBV DNA in mouse serum after mice were engrafted with PHH transfected with pgRNA ex vivo. GE stands for genome equivalents. LOQ stands for a limit of quantification. FIG. 2C shows Southern blot to detect HBV DNA in human hepatocytes isolated from an FNRG mouse (from panel A, marked by an encircled X) 93 days after injection with HBV pgRNA. kb stands for kilobases. FIG. 2D shows qPCR of HBV DNA in serum collected from naïve huFNRG mice infected with HBV from the serum of mice in FIG. 2A. GE stands for genome equivalents. LOQ stands for a limit of quantification. FIG. 2E shows immunostaining to detect HBcAg in HepG2-NTCP and HepG2 cells infected with HBV from the serum of mice in panel A. HepG2 cells that do not express NTCP as HBV receptor were used as a negative control. Percent infection is shown in the bar graph below.

FIGS. 3A, 3B, and 3C are a set of diagrams and photographs showing pan-genotype HBV replication from in vitro-transcribed HBV pgRNA. FIG. 3A shows an unrooted phylogenic tree of HBV genotypes A-H. Scale bar indicates substitutions per site. The unrooted phylogenic tree was generated using SeaView software. FIG. 3B shows qPCR of intracellular HBV DNA for HBV genotypes A-H. LOQ stands for limit of quantification. ETV stands for Entecavir. Filled bars represent mock treated, and open bars represent Entecavir treatment (10 μM). FIG. 3C shows chemiluminesence assay (CLIA) detects secreted HBsAg from HBV genotypes A-H six days post transfection with and without 10 μM ETV. Copies/well indicates copy number per well of a 6-well plate. LOQ stands for limit of quantification. ETV stands for Entecavir. Filled bars represent mock treated, and open bars represent Entecavir treatment (10 μM).

FIG. 4 shows the inhibitory effect of reverse-transcriptase inhibitors on WT HBV and the HBV viral variant M204V. The inhibitory effect of three reverse-transcriptase inhibitors, Entecavir (ETV), Tenofovir Disoproxil Fumarate (TDF), and Lamivudine (LAM) on WT HBV was compared. The inhibitory effect of LAM on the HBV viral variant M204V was also measured. The inhibition by the reverse-transcriptase inhibitors was determined as percent HBsAg by comparing the level of HBsAg in the presence and absence of the inhibitors.

FIGS. 5A, 5B, and 5C are a set of diagrams and photographs showing an exemplary process of selecting drug-resistant viral variants from in vitro-transcribed HBV pgRNA. FIG. 5A outlines a method to exploit the error-prone nature of T7 polymerase to generate a diverse population of RNA that can be selected in vitro. Depending on the selective pressure, reverse transcribed DNA in the cell, encapsidated DNA in the supernatant, or both, can be PCR amplified and sequenced directly or recloned for multiple rounds of selection to enrich for HBV variants. FIG. 5B shows 1% agarose gel of HBV DNA amplified two days post transfection from supernatants of cells untreated or treated with 40 or 400 μM LAM. FIG. 5C shows the statistical analysis of the identified mutations carried by the HBV viral variants selected in the presence of 400 μM LAM.

### DETAILED DESCRIPTION OF THE INVENTION

The disclosed methods for inducing HBV infection in cells are based on an unexpected discovery that inducing HBV infection with in vitro-transcribed HBV pgRNA achieved high signal-to-noise ratios for several assays tested, e.g., HBV qPCR and HBsAg CLIA. A major hurdle for HBV research and drug development is that signal-to-noise ratios for many assays that detect HBV proteins and nucleic acids are poor, and thus it, is difficult to predict drug-resistant viral variants prior to testing in the clinic.

Unlike viruses that replicate well in culture, HBV replicates poorly in culture. Most research studies and drug screens rely on only a few cell clones with integrated copies of the HBV genome to make viral stocks. Therefore, although there are ten HBV genotypes circulating in humans, most HBV studies include only a single genotype of wildtype (WT) virus. It is increasingly recognized, however, that studying multiple genotypes is important, since not all HBV genotypes respond equally well to anti-HBV therapies. Further, many in vitro HBV systems yield poor signal-to-noise ratios for virological assays, which complicate the interpretation of results and make it difficult to study the effects of specific viral mutations.

In general, to study multiple HBV genotypes and mutants, researchers either make genotype- or mutant-specific clonal virus producer cells by integrating an HBV template into cellular genomic DNA, or instead, by launching HBV infection with transfected plasmid DNA.

While these approaches are viable, working with multiple HBV producer cell clones is cumbersome and introduces variability. The alternative, using plasmid DNA to initiate HBV infection, produces high background signals since the large quantities of HBV proteins and nucleic acids produced from plasmid are often indistinguishable from those produced by virus replication.

Huang et al., in their 1991 paper, described experiments to infect cells by the RNA pregenome of an avian hepadnavirus, the duck hepatitis B virus (DHBV). However, no successful attempt to induce HBV infection in cells using pgRNA has been reported to date, in part, due to a number of differences between DHBV and HBV. First, DHBV belongs to the genus of Avihepadnavirus, whereas HBV belongs to the genus of Orthohepadnavirus. Second, Avihepadnaviruses (e.g., DHBV) lack a functional protein X, an essential protein for the infection of Orthohepadnaviruses (e.g., HBV). Third, Avihepadnaviruses do not produce surface protein M, only surface proteins S and L, whereas Orthohepadnaviruses produce all three surface proteins, S, M, and L. Lastly, a premise of pgRNA launch for infection is that this RNA is bicistronic to yield both core and polymerase proteins, both of which are essential for the establishment of virus replication. To produce the essential proteins for virus replication, DHBV likely utilizes a ribosome shunting mechanism, which is an efficient form of translation allowing translation to start in the absence of translation initiation factors. In contrast, HBV does not seem to utilize the ribosome shunting mechanism, but rather uses leaky scanning and ribosome reinitiation. Importantly, unlike DHBV which generates high and easily detectable levels of cccDNA in many systems that have been tested, including avian and human hepatoma cell lines, detection of HBV cccDNA remains challenging, particularly given its low level and a high background of other HBV DNA forms.

The present disclosure provides a method of initiating HBV infection in vitro and in vivo using in vitro-transcribed HBV pgRNA. This approach has several unique applications for basic research and drug discovery/development. For example, with this method, the signal-to-noise ratios for virological assays such as qPCR and HBsAg CLIA is excellent. The method can be used to launch infection in human liver chimeric mice and to compare multiple genotypes and virus mutants in vitro in a single cell type without the need for independent producer cell lines. This method can also be used to compare anti-HBV compounds and select for drug-resistant virus variants in vitro. Further, this last application should not only inform early decisions about which drugs to fast track for development, but also inform the design of next-generation compounds with better drug-resistance profiles and help define the viral protein or RNA sequence targeted by novel compounds with unknown mechanisms of action. Altogether, the RNA launch method may prove to be a valuable new tool to study basic HBV biology, identify novel anti-HBV agents, and inform drug development.

### I. INTRODUCING AN HBV GENOME IN A CELL

This disclosure provides a method for introducing an HBV genome in a cell. The method includes contacting a cell with a nucleic acid molecule including an HBV pgRNA from an HBV genotype and culturing the cell under a condition to initiate reverse transcription from the HBV pregenomic RNA and HBV replication.

pgRNA plays an essential role in viral genome replication. It has a size of about 3.5 kb and codes for reverse-transcriptase (polymerase), core, PreS, S, and X proteins (Seeger et al., 2000, Microbiol. Mol. Biol. Rev. 64:51-68). During the HBV life cycle, pgRNA is translated into HBV proteins or reverse-transcribed into HBV DNA. All the HBV proteins are important for HBV transcriptional regulation, viral genome packaging, reverse-transcription, and viral DNA recycling.

The nucleic acid molecule including an HBV pgRNA may be in vitro transcribed from an HBV DNA of an HBV genotype or chemically synthesized. The nucleic acid molecule harboring pgRNA may be single, double, or multiple stranded and may comprise modified or unmodified nucleotides or non-nucleotides or various mixtures and combinations thereof. The nucleic acid molecule may be a DNA-RNA hybrid. The nucleic acid may include a cap structure. The cap structure facilitates translation and protects the nucleic acid molecule from 5′exonuclease degradation and can help in delivery and/or localization within a cell. The cap can be present at the 5′-terminus (5′-cap) or at the 3′-terminus (3′-cap) or can be present on both termini. In one example, the nucleic acid molecule may be 3′-polyadenylated. The nucleic acid molecule may include a poly(A) tail containing non-adenine residues (e.g., cytosine (C), guanine (G), uracil (U)). For example, the poly(A) tail may include intermingled G residues to block 3′ exonuclease activity. In another example, the nucleic acid molecule may be 5′-capped. The 5′ cap may include a guanine nucleotide connected to RNA via an unusual 5′ to 5′ triphosphate linkage.

pgRNA may be polyadenylated by enzymatic modification. In one example, pgRNA may be polyadenylated using a poly(A) polymerase (e.g., E. coli Poly(A) Polymerase). In another example, pgRNA may be polyadenylated using a non-canonical poly(A) polymerase (e.g., TENT4A and TENT4B) to incorporate intermittent non-A residues (G, U, or C), which results in a heterogenous poly(A) tail. Alternatively, polyadenylation of pgRNA may be carried out by in vitro transcribing the pgRNA from a DNA template (e.g., plasmid, DNA segment) containing a 3′ poly(A) sequence (with or without intermittent non-A residues (G, U, or C)). In one example, the DNA template may contain a 3′ poly(A) sequence (with or without intermittent non-A residues (G, U, or C)) followed by a restriction site or a sequence containing the hepatitis delta ribozyme. The DNA template may be obtained by modifying HBV DNA using a molecular cloning technique (e.g., PCR, ligation) to incorporate a 3′ poly(A) sequence (with or without intermittent non-A residues (G, U, or C)). In one example, HBV DNA may be modified to incorporate a 3′ poly(A) sequence followed by a restriction site or a sequence containing the hepatitis delta ribozyme. Such a DNA template may be used to transcribe HBV pgRNA with a precise 3′ terminal sequence including a 3′ poly(A) sequence (with or without intermittent non-A residues (G, U, or C)), without further enzymatic modification to introduce the 3′ poly(A) sequence.

The HBV genotype includes all geographical genotypes of hepatitis B virus, particularly human hepatitis B virus, as well as variant strains of geographical genotypes of hepatitis B virus, which include HBV genotypes A, B, C, D, E, F, G, H, I, and J, and any subtype of the virus, such as subtypes adw, ayw, adr, and ayr. In some embodiments, the HBV genotype is genotype A, B, C, D, E, F, G, H, I, or J. The pgRNA may share substantial identity with any HBV genotype. For example, the pgRNA may include one or more mutations. The mutations can be introduced randomly along all or part of an HBV genotype coding sequence, such as by saturation mutagenesis.

For nucleic acids, the term “substantial identity” indicates that two nucleic acids, or designated sequences thereof, when optimally aligned and compared, are identical, with appropriate nucleotide insertions or deletions, in at least about 80% of the nucleotides, usually at least about 90% to 95%, and more preferably at least about 98% to 99.5% of the nucleotides. Alternatively, substantial homology exists when the segments will hybridize under selective hybridization conditions, to the complement of the strand.

For polypeptides, the term “substantial identity” indicates that two polypeptides, or designated sequences thereof, when optimally aligned and compared, are identical, with appropriate amino acid insertions or deletions, in at least about 80% of the amino acids, usually at least about 90% to 95%, and more preferably at least about 98% to 99.5% of the amino acids.

In vitro transcription of the HBV DNA may be carried out using any suitable RNA polymerase known in the art. Non-limiting examples of RNA polymerases may include T7, SP6, GH1, and T3 RNA polymerases. In some embodiments, the RNA polymerase is an error-prone T7 RNA polymerase. The HBV DNA template used for in vitro transcribing pgRNA may be single, double, or multiple stranded and may include modified or unmodified nucleotides or non-nucleotides or various mixtures and combinations thereof. The HBV DNA template may be a linear or circular DNA and may be a DNA fragment or a plasmid DNA containing a nucleic acid sequence encoding pgRNA. In some embodiments, the HBV DNA template is cccDNA.

In some embodiments, HBV pgRNA can be transcribed from a plasmid DNA library. The plasmid DNA library may contain HBV DNA templates generated by randomizing each codon in the HBV genome individually (primary library) or in combination with one other codon (secondary library). In comparison with the method using an error-prone T7 polymerase alone, the randomization method increases the level of diversity of the plasmid DNA library. T7 error generally introduces single nucleotide changes, and the likelihood of mutating two nucleotides in the same codon is very low. As a result, many codons are inaccessible through single nucleotide changes alone, thus limiting the diversity of genomes available for selection. Through randomization of each codon, targeted regions of the HBV genome are mutated to saturation. This increases the number of amino acids at each position and variants sampled, while at the same time significantly reducing the scale of experiments. Also, additional variations throughout the entire length of the HBV genome can be further introduced when error-prone T7 polymerase is used to transcribe the plasmid DNA library.

The cell to be in contact with the nucleic acid molecule may be a human cell or a higher primate cell. In some embodiments, the cell may be a HepG2 cell, a HepG2-NTCP cell, a Huh 7 cell, a Huh-7.5 cell, a Huh7.5-NTCP cell, a HepRG cell, a 293T cell, an HLC cell, or a human primary hepatocyte (PHH).

In some embodiments, the method further includes determining a level of HBV infection by quantifying the amount of one or more HBV proteins or HBV nucleic acids. HBV proteins include proteins produced (e.g., expressed) by HBV or mutant protein derivatives thereof, such as HBV antigens. HBV antigens may include, without limitation, HBV core proteins, such as Hepatitis B core protein (or HBcAg), Hepatitis B e antigen (or HBeAg), and envelope proteins, such as HBV surface antigen (or HBsAg). In some embodiments, the HBV proteins may include one or more HBV surface antigens, such as HBsAg. The HBV nucleic acids may be DNA or RNA and may be single, double, or multiple stranded. In some embodiments, HBV nucleic acids may include pgRNA, mRNA, S RNA, X RNA, PreC RNA, PreS1RNA, cccDNA, relaxed circular DNA (RC DNA), double-stranded linear DNA (DSL DNA), or fragments thereof.

The detection of HBV or its components in cells, cell lysates, or culture supernatant may be carried out by any suitable means known in the art, including any protein-based or nucleic acid-based detection means. The protein-based detection means may include, without limitation, spectrometry methods (e.g., HPLC, LC/MS) and antibody-dependent methods (e.g., ELISA, protein immunoprecipitation, immunoelectrophoresis, Western blot, protein immunostaining, chemiluminescent immunoassay (CLIA)). In some embodiments, CLIA is used as a detection means to quantify the amount of the HBV proteins. The nucleic acid-based detection means may include, without limitation, nucleic acid hybridization techniques, polymerase chain reactions (PCRs) (e.g., real-time PCR or qPCR), restriction fragment length polymorphism (RFLP), amplified fragment length polymorphism (AFLP), single-strand chain polymorphism (SSCP), amplification and mismatch detection (AMD), interspersed repetitive sequence polymerase chain reaction (IRS-PCR), inverse polymerase chain reaction (iPCR), reverse transcription polymerase chain reaction (RT-PCR), Northern blots, Southern blots, PCR sequencing, antibody procedures such as ELISA, and immunohistochemistry. In some embodiments, qPCR is used as the detection means to quantify the amount of the HBV nucleic acids.

In general, cells useful for this disclosure can be maintained and expanded in any suitable culture medium known in the art, with or without additional supplements. Such media include, without limitation, Dulbecco's Modified Eagle's Medium (DMEM), DMEM F12 medium, Eagle's Minimum Essential Medium, F-12K medium, Iscove's Modified Dulbecco's Medium, William's E Medium, and RPMI-1640 medium. Examples of supplements may include lipids, hormones, mammalian sera, amino acids, and trace elements.

### II. SCREENING FOR ANTI-HBV MODULATORS

This disclosure also provides a method for screening an HBV modulator for the ability to increase or decrease HBV infection. The method includes: (a) providing a cell having a nucleic acid molecule comprising an HBV pgRNA from an HBV genotype; (b) determining an amount of one or more HBV proteins or HBV nucleic acids in the presence of an HBV modulator; and (c) determining whether the HBV modulator increases or decreases HBV infection by comparing the amount to a reference amount that is obtained in the same manner except in the absence of the HBV modulator. The HBV modulator increases HBV infection if the amount is higher than the reference amount, and the HBV modulator decreases HBV infection if the amount is lower than the reference amount. The reference amount of HBV proteins or HBV nucleic acids in the absence of the HBV modulator may be measured using the same detection means as used for measuring the amount of HBV proteins or HBV nucleic acids in the presence of the HBV modulator. The reference amount of HBV proteins or HBV nucleic acids in the absence of the HBV modulator may be measured before, after and/or during the measurement of the amount of HBV proteins or HBV nucleic acids in the presence of the HBV modulator.

The increases or decreases of HBV infection refer to an elevation or reduction of the activity of HBV, HBV reverse transcriptase, the level of RNAs encoding one or more protein subunits of HBV, or the level of the HBV proteins compared to that observed in the absence of the HBV modulator. It does not necessarily refer to a total elimination of expression or activity. In some embodiments, the HBV modulator is an anti-HBV modulator (e.g., inhibitor, antagonist), having the ability to decrease HBV infection.

The above-described screening method can be used to screen one or more HBV modulators (e.g., agents, compounds) for anti-HBV activity. Examples of anti-HBV modulators may include a nucleic acid molecule (e.g., enzymatic nucleic acid molecule, antisense nucleic acid molecule, triplex oligonucleotide, dsRNA, ssRNA, RNAi, siRNA, aptamer, 2,5-A chimera), lipid, steroid, peptide, protein, allozyme, antibody, monoclonal antibody, humanized monoclonal antibody, small molecule (e.g., antiviral compounds), and/or isomers and analogs thereof, and/or a cell. HBV modulators may be used alone or in combination with another compound or therapy. In some embodiments, the HBV modulator may be a small molecule, an antibody, an aptamer, or an inhibitory nucleic acid molecule.

### III. SCREENING FOR HBV VARIANTS

The increasing reliance on chemical and immunological intervention in treating or preventing HBV infection is resulting in greater selective pressure for the emergence of variants of HBV which are resistant to the interventionist therapy. Due to the overlapping genomic structure of HBV, HBV variants may be directly or indirectly selected for by the use of chemical agents or vaccines. Further, it is important to be able to detect variant HBVs so that appropriate steps can be taken to modify a therapeutic protocol. This is also particularly important in the development of new therapeutic agents to be effective against known resistant variants of HBV and when cross-resistance develops within a family of chemically related anti-viral agents.

Accordingly, this disclosure also provides a method of identifying an HBV viral variant with altered sensitivity to an HBV modulator. The method includes: (a) contacting a cell with a nucleic acid molecule comprising pgRNA from an HBV genotype and (b) contacting the cell with an HBV modulator. Contacting the cell with the HBV modulator may be carried out before, during and/or after contacting the cell with a nucleic acid molecule comprising pgRNA. In some embodiments, the step of culturing the cell is performed in the presence of the HBV modulator.

The method further includes (c) culturing the cell to form a cell culture under a condition and for a period of time sufficient to initiate or induce reverse transcription from the HBV pgRNA and HBV replication, such that the HBV viral variant resistant to the HBV modulator is detected to replicate, express genetic sequences, or assemble or release virus or virus-like particles.

The method may further include (d) obtaining (e.g., isolating, extracting, harvesting) an HBV DNA from the cells or supernatant in the cell culture and (e) determining a mutation in the nucleotide sequence of the HBV DNA by sequencing the obtained HBV DNA. An HBV viral variant having the mutation is identified as the HBV viral variant with altered sensitivity to the HBV modulator.

The HBV DNA may be obtained from the cell or the culture supernatant by any suitable method known in the art, for example, by using a commercially available DNA extraction kit. An RNA polymerase binding site may be incorporated into the obtained HBV DNA by PCR, by ligation, or by cloning the HBV DNA into a vector containing the RNA polymerase binding site. The resulting HBV DNA may be further purified. The obtained HBV DNA may be of sufficient purity to serve as a template for in vitro transcription.

The present disclosure also provides a method for enriching mutations of an HBV viral variant. The method may include (f) obtaining a second nucleic acid molecule comprising an HBV pgRNA by in vitro transcription from the obtained HBV DNA and repeating steps (a) to (d), (f), and optionally step (e) one or more times. Alternatively, the method for enriching mutations of an HBV viral variant may include sub-culturing the cell culture in the presence of the HBV modulator one or more times.

The altered sensitivity includes an effect on viral infection, replication and/or assembly and/or release of a virus or virus-like particles or an effect on intermediary steps during the processes of infection, replication, assembly and/or release. In a preferred embodiment, the method may include determining whether the HBV viral variant is resistant or has a reduced sensitivity to an anti-HBV modulator. Resistance to an HBV modulator includes resistance to two or more chemically or functionally related modulators as may occur during the development of cross-resistance. For example, the method may include constructing a resistance test vector comprising the DNA of the HBV viral variant by using recombinant DNA technology (e.g., molecular cloning). The pgRNA of the HBV viral variant can be transcribed from the resistant test vector comprising the DNA of the HBV viral variant, and then introduced into a host cell (e.g., a HepG2 cell, a HepG2-NTCP cell, a Huh 7 cell, a Huh-7.5 cell, a Huh7.5-NTCP cell, a HepRG cell, a 293T cell, a HLC cell, or a human primary hepatocyte). The activity of the pgRNA of the HBV viral variant in initiating HBV replication is typically measured, in the presence and absence of an anti-HBV modulator, by determining an elevation or reduction of the activity of HBV, HBV reverse transcriptase, the level of RNAs encoding one or more protein subunits of HBV, or the level of the HBV proteins. The activity of the pgRNA of the HBV viral variant in initiating HBV replication is compared to that of the WT or parental HBV pgRNA to determine whether the HBV viral variant is resistant or has a reduced sensitivity to the anti-HBV modulator.

Optionally and/or additionally, the method may include determining whether or not the variant HBV has replicated, expressed genetic material, assembled, or been released in the presence of the HBV modulator, by subjecting the cells, cell lysates or culture supernatant to viral-detection means or viral-component-detection means. Methods for detecting HBV or its components (e.g., HBV proteins, HBV nucleic acids) in cells, cell lysates, or culture supernatant fluid are described above.

The HBV modulator may be an anti-HBV modulator, having the ability to reduce HBV infection. The HBV modulators may include antiviral agents (e.g., nucleosides) and immune modulators (e.g., interferons). For example, the HBV modulator may include, without limitation, Adefovir, Clevudine, Combivir, Coviracil, DAPD, DXG, L-FMAU, Dipivoxil, Emtricitabine (FTC), Entecavir (ETV), Epavudine, Epcitabine, Famvir, Lamivudine (LAM, 3TC or Epivir), Lobucavir, Penciclovir, Heptovir, Racivir, Trizivir, Tenofovir, Tenofovir Alafenamide (TAF), Tenofovir Disoproxil Fumarate (TDF), Telbivudine (TYZEKA), Zeffix, Intron A, L-DDA prodrug, HDP-p-Acyclovir, ara-AMP prodrugs, Thymosin alpha-1, Zadaxin, (−)-Carbovir, Hammerhead ribozymes, Genevax, Pres1/s2 vaccine, Hepagene, HBV immunoglobulin, Nabi-HBV, Glycosidase inhibitors, nonyl-DNJ, or interferon family members, without limitation, including alpha, beta, gamma or lambda. In another example, the HBV modulator may also include HBV vaccine. The HBV modulator may include human monoclonal antibodies directed against HBV, therapeutic vaccine strategies (e.g., live, attenuated, and replication-incompetent virus or killed, inactivated virus), and any other antiviral composition in use or in development for use to treat HBV infection.

In some embodiments, the HBV modulator may be a small molecule, an antibody, an aptamer, or an inhibitory nucleic acid molecule. HBV modulator may be a nucleoside analog or a non-nucleoside analog. For example, the HBV modulator may be a non-nucleoside reverse transcriptase inhibitor and/or a non-nucleoside DNA polymerase inhibitor. In some embodiments, the nucleoside analog is 3TC, PMEA or PCV. In some embodiments, the HBV modulator is an immunointeractive molecule, such as an antibody. In some embodiments, the HBV modulator may include inhibitors targeting the formation and/or replication of HBV, such as a capsid assembly inhibitor, a viral polymerase inhibitor, or a reverse-transcriptase inhibitor.

In some embodiments, the HBV viral variant is capable of replicating in the presence of an HBV modulator, which inhibits or reduces infection, replication, or assembly of a reference/wild-type HBV. An HBV viral variant generally contains a single or multiple nucleotide substitution, addition and/or deletion or truncation mutation in the viral genome and a corresponding single or multiple amino acid substitutions, addition and/or deletion or truncation in a viral peptide, polypeptide or protein. In some embodiments, the HBV viral variant comprises an altered HBV DNA polymerase, and altered HBV precore promoter or basal core promoter, an altered HBsAg, or a combination thereof.

In some embodiments, the mutation that in the nucleotide sequence of the HBV DNA that gives rise to altered sensitivity to the HBV modulator is determined by sequencing the obtained HBV DNA. The identification of viral variants enables the production of vaccines comprising particular recombinant viral components such as polymerases or envelope genes PreS1, PreS2, S encoding for L, M, S proteins. Rational drug design may also be employed to identify or generate therapeutic molecules capable of interacting with an altered polymerase or envelope genes PreS1, PreS2, S encoding for L, M, S proteins or other components of the HBV.

In some embodiments, sequencing methods may include next-generation sequencing (NGS) methods, in which clonally amplified DNA templates or single DNA molecules are sequenced in a massively parallel fashion. The advent of next-generation sequencing (NGS) technologies, which allow for sequencing entire genomes in a relatively short time, has provided the opportunity to analyze genetic material without the risks associated with invasive sampling methods.

### IV. GENERATING ANIMAL MODELS

This disclosure also provides a method of introducing (e.g., injecting) HBV infection in an animal model. The method can be used to establish HBV animal models for screening compounds and/or therapies directed against HBV. The method includes introducing (e.g., injecting) into the liver of an animal model a nucleic acid molecule comprising an HBV pgRNA from an HBV genotype. In some embodiments, the animal model is a human liver chimeric mouse. The human liver chimeric mouse may have a high degree of human hepatocyte engraftment. In some embodiments, the method may also include introducing (e.g., injecting) into the larger liver lobe of the animal model a nucleic acid molecule comprising an HBV pgRNA from an HBV genotype.

Alternatively, HBV infection in an animal model can be induced by introducing (e.g., transplanting) HBV-infected liver tissue or cells to the liver of the animal model. The HBV-infected liver tissue or cells may be obtained from an animal model or cells with HBV infection induced by the above-described methods. A chimeric mouse model for HBV infection may be generated by introducing HBV-infected human liver tissue or hepatocytes to the liver of a mouse model. In some embodiments, the mouse model is an immunodeficient mouse. The cell used for inducing HBV infection may include, without limitation, a HepG2 cell, a HepG2-NTCP cell, a Huh 7 cell, a Huh-7.5 cell, a Huh7.5-NTCP cell, a HepRG cell, a 293T cell, an HLC cell, or a human primary hepatocyte (PHH). In some embodiments, the method may include injecting, for example by subcutaneous injection, HBV infected or HBV pgRNA transfected PHH cells into the mouse under conditions suitable for the propagation of PHH cells in the mouse. For example, PHH cells can be suspended in Dulbecco's PBS solution supplemented with calcium and magnesium. In some embodiments, PHH cells may be selected for antibiotic resistance before being introduced into the mouse model.

### V. KITS

This disclosure further provides a kit for introducing an HBV genome in a cell. The kit includes a nucleic acid molecule comprising an HBV pgRNA from an HBV genotype. The nucleic acid molecule including an HBV pgRNA may be in vitro transcribed from an HBV DNA of an HBV genotype or chemically synthesized. The nucleic acid molecule comprising pgRNA may be single, double, or multiple stranded and may include modified or unmodified nucleotides or non-nucleotides or various mixtures and combinations thereof. The nucleic acid molecule may be a DNA-RNA hybrid. The nucleic acid may include a cap structure. The cap can be present at the 5′-terminus (5′-cap) or at the 3′-terminus (3′-cap) or can be present on both termini. In one example, the nucleic acid molecule may be 3′-polyadenylated. The nucleic acid molecule may include a poly(A) tail containing non-adenine residues (e.g., cytosine (C), guanine (G), uracil (U)). For example, the poly(A) tail may include intermingled G residues to block 3′ exonuclease activity. In another example, the nucleic acid molecule may be a 5′-capped. The 5′ cap may include a guanine nucleotide connected to mRNA via an unusual 5′ to 5′ triphosphate linkage.

The pgRNA may belong to any HBV genotype including all geographical genotypes of hepatitis B virus, particularly human hepatitis B virus, as well as variant strains of geographical genotypes of hepatitis B virus, which include HBV genotypes A, B, C, D, E, F, G, H, I, and J, and any subtype of the virus, such as subtypes adw, ayw, adr and ayr. In some embodiments, the pgRNA used in the kit belongs to HBV genotype A, B, C, D, E, F, G, H, I, or J. The pgRNA may share substantial identity with any HBV genotype (e.g., at least about 80% of the nucleotides, usually at least about 90% to 95%, and more preferably at least about 98% to 99.5% of the nucleotides). In some embodiments, the pgRNA may include a patient-derived sequence. The patient-derived sequence may be determined from a sample (e.g., an HBV DNA segment) obtained from a patient infected by HBV.

Alternatively, a kit for introducing HBV genome in a cell may include a DNA molecule comprising a binding site for an RNA polymerase and a DNA sequence encoding an HBV pgRNA from an HBV genotype. In some embodiments, the kit may include a plurality of DNA molecules generate by randomizing individual codons, each of the plurality of DNA molecules includes a binding site for an RNA polymerase and a DNA sequence encoding an HBV pgRNA from an HBV genotype. In some embodiments, the binding site may include a promoter region that is typically located directly upstream or at the 5′ end of the transcription initiation site. In some embodiments, the promoter region may include a T7 promoter. In some embodiments, the kit further includes an RNA polymerase, optionally one or more nucleoside triphosphates, and a buffer. Non-limiting examples of RNA polymerases may include T7, SP6, GH1, and T3 RNA polymerases. In some embodiments, the RNA polymerase is an error-prone RNA polymerase, such as error-prone T7 RNA polymerase.

The DNA molecule encoding the HBV pgRNA may be single, double, or multiple stranded and may comprise modified or unmodified nucleotides or non-nucleotides or various mixtures and combinations thereof. The DNA molecule may be a linear or circular DNA and may be a DNA fragment/segment or a plasmid DNA containing a nucleic acid sequence encoding pgRNA. In some embodiments, the DNA molecule may be cccDNA, relaxed circular DNA (RC DNA), or double-stranded linear DNA (DSL DNA). In some embodiments, the DNA molecule may include a DNA segment generated with an error-prone DNA polymerase (e.g., Pol V encoded by UmuD'C, Pol IV encoded by DinB).

The kits for introducing HBV genome in a cell may include instructions for use, and may further contain various reagents, solvents, diluents, and/or pharmaceutically acceptable preservatives. The nucleic acid molecule or the DNA molecule may be in a lyophilized form or provided with an acceptable carrier, preferably an aqueous carrier, with the nucleic acid molecule or the DNA molecule comprising an HBV pgRNA dissolved or suspended therein. A variety of aqueous carriers may be used, e.g., water, buffered water, 0.8% saline, 0.3% glycine, hyaluronic acid and the like.

### VI. DEFINITIONS

As used in this document, the singular forms “a,” “an,” and “the” include plural references unless the context clearly dictates otherwise. Unless defined otherwise, all technical and scientific terms used here.

The term “and/or” means any one of the items, any combination of the items, or all of the items with which this term is associated.

The compositions of the present invention can comprise, consist essentially of, or consist of the claimed ingredients. The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the term “HBV” refers to mammalian hepatitis B virus, including human hepatitis B virus. The term encompasses all geographical genotypes of hepatitis B virus, particularly human hepatitis B virus, as well as variant strains of geographical genotypes of hepatitis B virus.

As used herein, the term “HBV nucleic acid” refers to any nucleic acid encoding an HBV protein. For example, an HBV nucleic acid includes, without limitation, any viral DNA sequence encoding an HBV genome or portion thereof, any RNA sequence transcribed from a viral DNA including any mRNA sequence encoding an HBV protein.

As used herein, the term “nucleic acid molecule” refers to a molecule having nucleotides. The nucleic acid can be single, double, or multiple stranded and may include modified or unmodified nucleotides or non-nucleotides or various mixtures and combinations thereof. The nucleic acid molecule may be a DNA-RNA hybrid.

As used herein, the term “HBV protein” refers to any protein produced by HBV or a mutant protein derivative thereof, comprising sequence expressed and/or encoded by the HBV genome. The term encompasses various HBV antigens, including core proteins, such as Hepatitis B core antigen (or HBcAg), Hepatitis B e antigen (or HBeAg), and envelope proteins, such as HBV surface antigen (or HBsAg).

As used in herein, the term “cell” is used in its usual biological sense and does not refer to an entire multicellular organism, e.g., specifically does not refer to a human. The cell may be a eukaryotic cell (e.g., a mammalian cell).

As used herein, the term “transfected” or “transfection” refer to introducing exogenous DNA or RNA into a host cell any suitable means known in the art (e.g., chemical-based transfection, cell squeezing, electroporation, magnetofection, nanoparticle systems). Host cells may also be cotransfected with a second recombinant DNA molecule comprising a DNA sequence encoding for expression of an amino acid sequence corresponding to a substantial portion or all of the amino acid sequence of the HBV S peptide.

As used herein, the term “modulator” refers to a molecule capable of modulating (up-regulating or down-regulating) the expression of the gene, or level of RNAs encoding one or more protein subunits or components, or activity of one or more proteins, such that the expression, level, or activity is greater than or less than that observed in the absence of the modulator.

As used herein, the term “inhibiting HBV” refers to reducing the level or expression of an HBV mRNA, DNA and/or protein (e.g., reverse transcriptase, protein subunits of HBV), virus assembly, formation, and/or secretion. For example, HBV may be inhibited in the presence of a nucleoside analog targeting HBV, as compared to expression of HBV mRNA, DNA and/or protein levels in the absence of the nucleoside analog.

As used herein, the term “variant” is used in its broadest context and includes mutants, derivatives, modified and altered forms of an HBV relative to a reference HBV. A variant generally contains a single or multiple nucleotide substitution, addition and/or deletion or truncation mutation in the viral genome and a corresponding single or multiple amino acid substitutions, addition and/or deletion or truncation in a viral peptide, polypeptide or protein.

As used herein, the term “sequencing” herein refers to a method for determining the nucleotide sequence of a polynucleotide, e.g., genomic DNA. Preferably, sequencing methods include, as non-limiting examples, next generations sequencing (NGS) methods, in which clonally amplified DNA templates or single DNA molecules are sequenced in a massively parallel fashion (e.g., as described in Volkerding et al. Clin Chem 55:641-658, 2009; Metzker M Nature Rev 11:31-46; 2010).

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism such as a non-human animal.

The terms “decrease,” “reduced,” “reduction,” “decrease,” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Unless defined otherwise, all technical and scientific terms used herein have the same meaning as commonly understood by one of ordinary skill in the art to which this invention belongs. Although any methods and materials similar or necessarily to those described herein can also be used in the practice or testing of the present invention, the preferred methods and materials are now described. All publications mentioned herein are incorporated herein by reference in their entireties.

### VII. EXAMPLES

### Example 1

This example describes the materials and methods to be used in the subsequent examples.

Materials

Plasmids

All plasmid sequences used in this study have been uploaded to GenBank. Briefly, HBV pgRNA sequences were cloned into the pGEM-3Z plasmid backbone immediately downstream of a T7 promoter. The genotype A sequence has been previously described as adw2 serotype (Valenzuela et al., Animal Virus Genetics 1980, 57-70). Genotypes B-H were cloned into the same backbone.

The HBV-YMHD Polymerase mutant was made in the genotype A backbone. As an additional control for transfection efficiency and qPCR, T7-RFP by replacing nucleotides 1-1005 of genotype A HBV with nucleotides encoding tagRFP were generated.

Cells

HepG2-NTCP cells have been previously described, and Huh-7.5-NTCP cells were made with the same protocol (Michalidis et al., Sci Rep. 2017, November 30:7(1):16616). Cells were maintained in Dulbecco's Modified Eagle Medium (DMEM, Fisher Scientific, cat. #11995065) supplemented with 0.1 mM nonessential amino acids (NEAA, Fisher Scientific, cat. #11140076) and 10% hyclone fetal bovine serum (FBS, HyClone Laboratories, Lot. #AUJ35777). HepG2-NTCP cells were maintained on collagen-coated plates.

### Liver Chimeric Mice

Immunodeficient Fah^{-/-} NOD Rag1^{-/-} Il2rg^{null} (FNRG) female mice were cycled off the protective drug nitisinone (Yecuris, cat. #20-0028) to induce murine liver injury and transplanted with 500,000 primary human hepatocytes (Lonza, cat. #HUCPM) per mouse (Azuma et al., Nat Biotechnol. 2007 August: 25(8):903-910; de Jone et al., Sci Transl Med. 2014 September 17:6(254)) Human engraftment was monitored by human albumin (hAlb) quantification in mouse serum by ELISA (Bethyl Labs, cat. #E88-129) (Billerbeck et al., J Hepatol. 2016 August; 65(2):334-43). Humanized FNRG (huFNRG) mice engrafted to ˜10 mg/ml of hAlb were used for pgRNA injection.

Methods

In Vitro Transcription & Polyadenylation

10 μg of plasmid DNA was linearized by digestion with NotI-HF (genotypes A and RFP control) or XmnI (genotypes B-H). Linearized DNA was purified with the MinElute PCR purification kit (Qiagen, cat. #28004) according to the manufacturer's instructions, diluted to 0.5 μg/μl in EB buffer, and used as template for in vitro transcription using the mMESSAGE mMACHINE® T7 Transcription kit (Fisher Scientific, cat. #AM1344). Transcription was performed in 40 μl reaction volume containing 1× NTP/cap, 6 mM extra GTP, 2 μg linearized DNA and 4 μl T7 Enzyme mix. To achieve maximal yield, the reaction was incubated at 37° C. for 4 h followed by 15 min DNase treatment at 37° C. The reaction was stopped by adding 1 ml of Trizol (Fisher Scientific, cat. #15596026). Next, 200 μl of chloroform was added, and the mixture was transferred to a MaXtract High-Density tube (Qiagen, cat. #129056) and centrifuged at 12,000×g for 10 min at 4° C. The aqueous supernatant was transferred to a new 1.5 ml tube and mixed with an equal volume of 100% ethanol then loaded onto an RNeasy mini kit column (Qiagen, cat. #74014). The RNAs were purified following the manufacturer's instructions, including the optional on-column DNase digestion (Qiagen, cat. #79254). The average RNA yield was ˜45-70 μg per reaction.

RNAs were polyadenylated using E. coli Poly(A) Polymerase (NEB, cat. #M0276). Each 10 μg of RNA was polyadenylated in a 20 μl reaction at 37° C. for 30 min with 1× buffer, 0.2 mM ATP, 20 U Superase. In and 40 U E. coli Poly(A) Polymerase. Optionally, RNA may be polyadenylated using non-canonical poly(A) polymerases (e.g., TENT4A and TENT4B) to incorporate intermittent non-A residues (G, U, or C), which results in a heterogenous poly(A) tail. The polyadenylated RNAs were then purified using the RNeasy mini kit, this time without the optional DNase treatment step.

RNA Transfection

HepG2-NTCP or Huh-7.5-NTCP cells were seeded at 2.5×10\hat{ }5 cells per well in six-well plates two days before transfection. The media was changed to 2 ml DMEM containing 1.5% FBS and 0.1 mM NEAA just before transfection. For each transfected well, 0.5 μg (for HepG2-NTCP) or 1 μg (for Huh-7.5-NTCP) RNA was mixed with 5 μl Lipofectamine™ 2000 (Fisher Scientific, cat. #11668019) in 500 μl Opti-MEM Reduced Serum Medium (Fisher Scientific, cat. #51985034) and incubated at room temperature for 20 min. The mixture was then added to cells and spinoculated by centrifugation at 1000×g for 30 min at 37° C. Six hours later the media was removed and replaced with DMEM containing 10% FBS and 0.1 mM NEAA.

Analysis of HBV Replication Intermediates

HBeAg and HBsAg were quantified using a chemiluminescence immunoassay (CLIA) kit (Autobio Diagnostics Co., Zhengzhou, China) according to the manufacturer's instructions. To quantify HBV DNA, total DNA was extracted from infected or RNA-transfected cells using QIAamp DNA Blood Mini Kit (Qiagen, cat. #51106) and HBV DNA was detected by qPCR as previously described (Michalidis et al., Sci Rep. 2017, November 30:7(1):16616). For each experiment, cells were transfected with T7-RFP as a negative control and DNA quantified from these cells was used to define the background. For Southern blot, HBV cccDNA was enriched by Hirt extraction as previously described (Cai et al., Methods Mol Biol. 2013; 1030:151-161). RNA in the extract was digested in 400 μl 1× Cutsmart buffer (NEB) with 40 μg RNase A (Thermo Scientific, cat. #EN0531) at 37° C. for 2 h. The reaction was stopped by adding 200 μg of proteinase K (Fisher Scientific, cat. #25530015) for 30 min at 37° C. followed by phenol/chloroform extraction. DNA was ethanol precipitated and dissolved in 25 μl TE (Tris 10 mM, EDTA 1 mM). DNA extracted two, four, and six days after transfection were combined and split into three separate tubes. One tube was left untreated, and the other two were heated to 88° C. for 5 min to convert linear and relaxed circular (RC) DNA to single-stranded (SS) DNA, leaving covalently closed circular DNA (cccDNA) intact. After cooling on ice, one of the heated tubes was treated with 40 U EcoRI in 30 μl 1× EcoRI buffer at 37° C. for 2 h to linearize cccDNA. All samples were adjusted to 1× EcoRI buffer before being loaded in a 1.2% agarose TAE gel and run overnight at 45V-50V at 4° C. DNA was then transferred to a Hybond-XL membrane (Fisher Scientific, cat. #RPN303N) for 36-48 h. 32P-labeled hybridization probes were prepared with the Prime-It II Random Primer Labeling Kit (Agilent Technologies, cat. #300385) using linearized 2× HBV DNA as a template as previously described (Doitsh G, Shaul Y. Virology 2003, May 10; 309(2) 339-49). Probes were hybridized in 10 ml of EKONO™ Hybridization Buffer (G-Biosciences, #768-160) overnight at 65° C. The membrane was washed as described (Cai et al., Methods Mol Biol. 2013; 1030:151-161) and the hybridized bands were visualized by phosphorimager.

RNA Injection Into Liver Chimeric Mice

huFNRG mice were anesthetized with isoflurane, and an 8-12 mm skin incision was made under the left sternal margin. After opening the peritoneum, the large liver lobe was mobilized out of the peritoneal cavity. Prior to injection a 32G syringe (Hamilton, cat. #90032) was cleaned with RNase AWAY (Thermo Scientific, cat. #7002) and distilled water. Then pgRNA suspended in 200 μl PBS−/− (Fisher Scientific, cat. #14190144) was injected into the large liver lobe in 8-10 injections. The injection site was cauterized, the liver was mobilized back, and the peritoneum was closed with 4.0 Vicryl sutures (Ethicon, cat. #J663H). The skin was closed with wound clips, and mice received meloxicam (Norbrook) for postoperative analgesia.

For viral infection, huFNRG mice were challenged through the tail vein with cryopreserved HBV-positive serum obtained from the mice originally injected with pgRNA. For serum tests, mice were bled retroorbitally, and serum was obtained after centrifugation. hAlb, Southern blot of Hirt DNA and viral DNA quantification were performed as described above and previously (Billerbeck et al., J Hepatol. 2016 August; 65(2):334-43).

HBV Infection

HepG2-NTCP cells were seeded in either 96-well collagen-coated plates or 35 mm glass bottom collagen-coated dishes (MatTek, cat #. P35GCOL-1.5-14-C) using conditions described before (Michalidis et al., Sci Rep. 2017, November 30:7(1):16616). Briefly, confluent cultures were treated with 2% DMSO for 24 h prior to infection and were subsequently infected with serum from viremic mice at indicated GEQ/cell in medium supplemented with 4% polyethylene glycol 8000 and 2% DMSO. Virus inoculum derived from HepDE19 cell supernatant was used as a positive control. HepG2 cells that do not express NTCP were used as a negative control. One day after infection, cells were washed extensively, and infection was terminated seven days later. Cells were fixed and stained using a rabbit anti-HBV core antibody (Austral Biologicals, cat. #HBP-023-9) as described before (Michalidis et al., Sci Rep. 2017, November 30:7(1):16616).

Circseq Analysis of In Vitro-Transcribed pgRNA

Circseq allows for highly accurate sequencing of viral RNA populations (Acevedo et al., 2014 January 30:505(7485):686-90). In summary, short fragments of the in vitro-transcribed pgRNA of HBV WT genotype A were circularized and converted to DNA by rolling reverse transcription to yield tandemly repeated cDNAs. Randomly distributed errors generated during library preparation were filtered out by computational mapping of repeats.

The repeat fragments were first aligned to generate a consensus read and then the frequency of mutants present in the in vitro-transcribed pgRNA population was then calculated by mapping these reads the original sequence.

Enriching Lamivudine-Resistant Viral Variants

To enrich mutations that confer Lamivudine (LAM) resistance, 2.5×10\hat{ }5 Huh-7.5-NTCP cells were seeded two days before pgRNA transfection as described above. The cells were treated with 400 μM LAM 24 h before RNA transfection. Transfection was performed, and cells were maintained in the presence of 400 μM Lamivudine. The supernatant was collected two days after transfection and concentrated with Amicon-Ultra-0.5 ml 100 kDa centrifugal filters (Millipore, cat. #UFC510096) to a volume of 200 μl. HBV DNA was then extracted using the QIAamp MinElute Virus Spin Kit (Qiagen, cat. #57704) and amplified by two PCR reactions prior to sequencing. The PCR products from both rounds were separated in a 1% TAE agarose gel and purified by gel extraction (Qiagen, cat. #28704). The first PCR used primers RU-O-26880 and RU-O-24242 to get full-length HBV sequence. The second PCR used primers RU-O-26264 and RU-O-26265 to amplify the RT region of HBV. DNA from the second PCR was submitted to the CCIB DNA Core Facility, Massachusetts General Hospital (Cambridge, Mass., USA) for high throughput sequencing.

DNA Sequencing Data Analysis

Sequences for each sample were aligned using tophat2 with a maximum of two mismatches allowed. The Rsamtools package was used to extract counts for each nucleotide at each position of the alignment, ignoring sequence with a quality score lower than 13. For paired sample sets (one sample generated in the absence of LAM and generated in the presence of 400 μM Lamivudine), one-sided Fisher's exact tests on count data for each possible mutation was performed to obtain p-values for the enrichment of each mutation in the presence of Lamivudine. Mutations were ranked according to p-values obtained from weighted z-transformation tests across all of the independent paired sample sets using the metap package. Source code for variant analysis can be obtained at Github site (https://github.com/yyp777/HBV-drug-resistance-mutation).

### Example 2

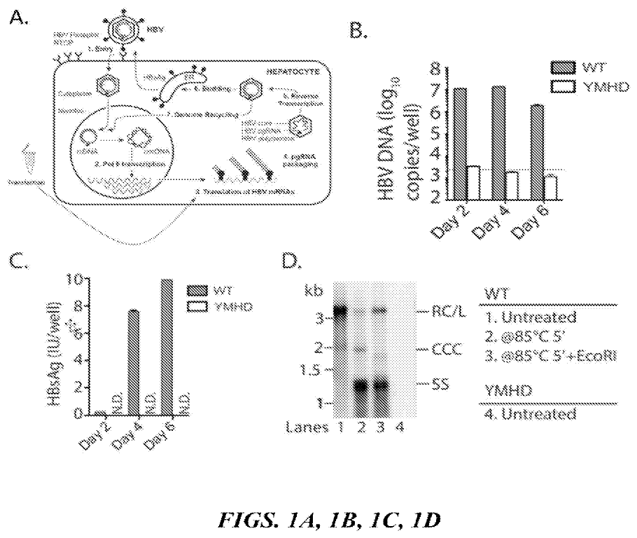

To launch HBV infection with RNA, Huh-7.5-NTCP and HepG2-NTCP cells were transfected with the 3.5 kb HBV pregenomic RNA (pgRNA). pgRNA was chosen because it is the template for translation of the HBV core and polymerase (Pol) proteins, both of which are required for reverse transcription, and it is also the template for reverse transcription which leads to cccDNA (FIG. 1A).

It was found that transfecting Huh-7.5-NTCP cells (FIG. 1B) and HepG2-NTCP cells with in vitro-transcribed HBV pgRNA gives rise to reverse-transcribed HBV DNA. As expected, the DNA signal is HBV Pol-dependent, as a catalytic site mutation (YMHD) decreases the DNA signal up to 10,000 fold. The Huh-7.5-NTCP yielded more DNA than HepG2-NTCP cells, and thus the studies using Huh-7.5-NTCP cells were continued.

Next, whether the reverse-transcribed DNA in Huh-7.5-NTCP cells was functional was tested. If the reverse-transcribed DNA in Huh-7.5-NTCP cells was functional, it should “recycle” to the nucleus, serve as a template for Pol II transcription, and give rise to additional HBV mRNAs (see lifecycle, FIG. 1A). Consistent with this outcome, FIG. 1C shows that HBsAg is produced in increasing quantities over time by cells transfected with wild-type (WT), but not mutant, HBV pgRNA. Superior to other HBV systems, the signal-to-noise ratio with this assay is excellent, as there is zero HBsAg background signal from mutant HBV pgRNA.

While these results indicated that HBV DNA was transcribed, it was possible that the template for transcription was linear episomal or integrated HBV DNA rather than the authentic template for HBV mRNA transcription, cccDNA. To test this possibility, Southern blot on Hirt-extracted DNA to test whether cccDNA was formed was performed. FIG. 1D shows that cccDNA was indeed formed in these cells. While this does not rule out the possibility that linear episomal or integrated DNA contribute to HBV mRNA transcription and HBsAg production, it nevertheless proves that cccDNA was made. Together, these results suggested that HBV pgRNA may be infectious.

Injecting RNA directly into the liver is a common strategy used to launch infection of positive-stranded hepatotropic RNA viruses in animal models. This, however, has not been shown for HBV, a dsDNA virus. Whether injecting HBV pgRNA into the livers of human liver chimeric mice (the FNRG mouse model) could initiate HBV infection was tested. For this, mice with a high degree of human hepatocyte engraftment, estimated by measuring human albumin in serum were used.

### Example 3

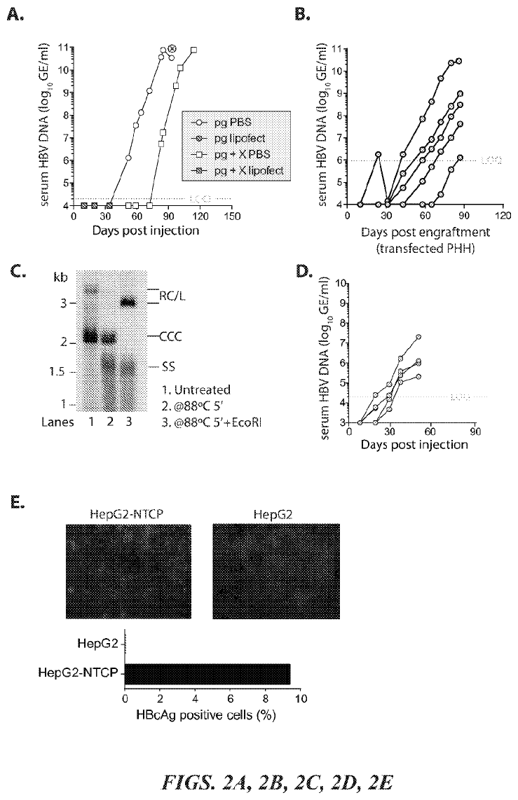

To launch HBV infection in an animal model, in vitro-transcribed HBV pgRNA was either injected into the livers of human liver chimeric mice or transfected into PHH ex vivo and the transfected cells were then engrafted into human liver chimeric mice. FIG. 2A shows that injecting in vitro-transcribed HBV pgRNA into the livers of human liver chimeric mice initiates robust HBV infection. FIG. 2B shows that engrafting PHH that were transfected with in vitro-transcribed HBV pgRNA into the livers of human liver chimeric mice initiates robust HBV infection. In both cases, the onset of viremia was slow, appearing approximately six weeks post injection, then skyrocketed, similar to the kinetics of HBV infection in humans To further demonstrate that injected HBV pgRNA produced authentic HBV infection, serum from an HBV positive mouse shown in FIG. 2A was used to infect naïve mice. FIG. 2D shows that all four infected mice became viremic. As further proof, the liver from a viremic mouse in FIG. 2A was harvested to extract DNA and a Southern blot was performed to detect cccDNA. FIG. 2C shows that the liver from this mouse contained an abundance of cccDNA.

For reasons that are unclear, it has been difficult to infect human liver chimeric mice with clinical samples or with tissue culture-derived HBV. Once infection is established, however, virus from mouse serum readily infects naïve mice. Next, whether virus from the blood of a mouse injected with HBV pgRNA could infect naïve human liver chimeric mice and cultured HepG2-NTCP cells was tested. It was found that virus in mouse blood infected both naïve human liver chimeric mice (FIG. 2D) and cultured HepG2-NTCP cells (FIG. 2E). The virus from the inoculum was then sequenced. It was found that the sequence was identical to that of the template used for in vitro-transcription (data not shown), indicating that viral adaptation was not required for efficient infection and replication in these mice.

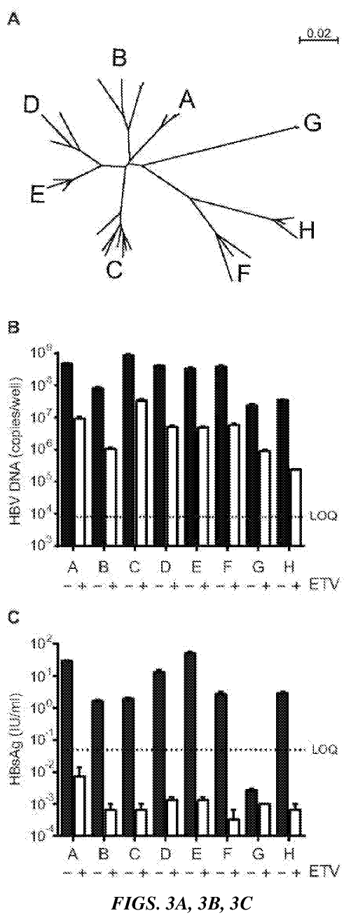

Thus far, the results were obtained using the genotype A virus. However, there are at least ten HBV genotypes, several of which are highly prevalent in various parts of the world (FIG. 3A). Next, whether the RNA launch method could be used to study multiple HBV genotypes in vitro was tested. To this end, representative pgRNA encoding sequences for HB V genotypes B-H downstream of a T7 promoter were cloned, from which pgRNAs were in vitro transcribed. Following transfection of Huh-7.5-NTCP cells, pgRNA from genotypes B-H, like genotype A pgRNA, also yielded HBV DNA (FIG. 3B) and HBsAg (FIG. 3C). As shown above, the signal-to-noise ratios for both assays were excellent. This demonstrates that the RNA launch method can be applied to study multiple HBV genotypes and viral mutants without the high background signal typical of plasmid transfection approaches or the variability imposed by producing virus from multiple clonal producer cells.

### Example 4

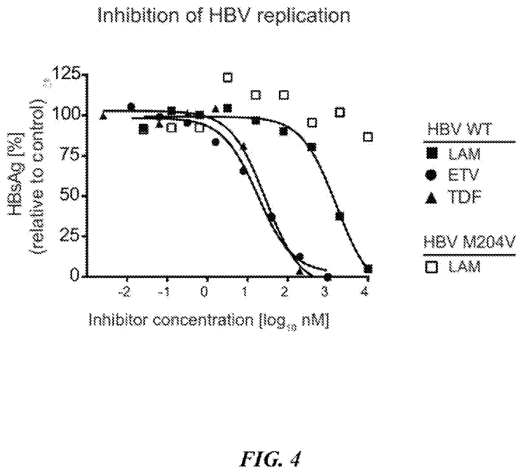

Next, whether the RNA launch method could be used to compare the potency and effectiveness of anti-HBV inhibitors was tested. As shown in FIG. 4, the inhibitory effect, on WT HBV, of three reverse-transcriptase inhibitors known to inhibit HBV: Entecavir (ETV), Tenofovir Disoproxil Fumarate (TDF), and Lamivudine (LAM), were compared. All three drugs inhibited HBV replication in a dose-dependent manner pgRNA with a mutation (M204V) known to confer resistance to LAM was also tested in this study. As shown in FIG. 4, it was found that M204V pgRNA was resistant to LAM. These results indicate that LAM inhibition is specific and not due to cellular toxicity at higher drug concentrations. Overall, these results demonstrate that the RNA launch method can be used to study the efficacy of anti-HBV compounds in a simple and easy-to-use assay. The results also suggest that the method could potentially be used as a new screening tool to discover novel HBV inhibitors.

### Example 5

Next, whether the RNA launch method could be used to select drug-resistance mutations was tested. Drug-resistance mutations can help define drug resistance barriers and identify viral targets of novel compounds. This information can inform early decisions in drug development pipelines such as which compounds to pursue for further development and also facilitate the design of second and third-generation inhibitors with better drug-resistance profiles. However, selecting drug resistance mutations for HBV has been difficult since infection frequency is low with current in vitro systems and the virus spreads too poorly for drug-resistant variants to emerge. Indeed, it was not known that LAM would have a low barrier to resistance until after the drug had been used in the clinic where it was later found that 70% of patients treated with LAM harbor LAM-resistant virus after five years of therapy.

### Example 6

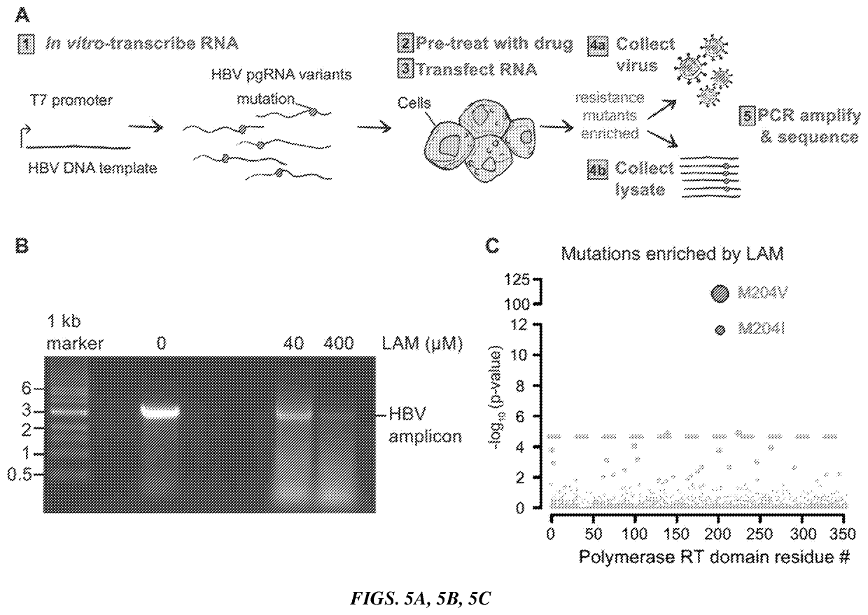

Several features of the RNA launch approach allow for enrichment of HBV drug-resistant variants. For one, unlike protocols that initiate infection with plasmid DNA, sequence diversity is inherent in the pgRNA population. This is because T7 polymerase, used to transcribe HBV pgRNA, has an error rate of ˜10⁻⁴ and therefore produces a diverse mixture of sequence variants immediately available for selection. Also, since transfecting pgRNA bypasses the entry step, cells likely sample more genomes than they would with other HBV systems that initiate infection with virus particles. Further, since drug-resistant genomes can be enriched by PCR without contamination from input plasmid or viral DNA, it should be possible to identify mutations that confer drug resistance by deep sequencing even without robust virus replication and spread. The cartoon schematic in FIG. 5A outlines this process.

Alternatively and/or additionally, plasmid DNA libraries with high diversity are generated by randomizing each codon in the HBV genome individually (primary library) or in combination with one other codon (secondary library). In comparison with the above method using an error-prone T7 polymerase, the randomization method increases the diversity of plasmid DNA libraries. T7 error generally introduces single nucleotide changes, and the likelihood of mutating two nucleotides in the same codon is very low. Many codons, however, are inaccessible through single nucleotide changes alone, thus limiting the diversity of genomes available for selection. Through randomization of each codon, targeted regions of the HBV genome are mutated to saturation. As a result, the number of amino acids at each position and variants sampled is increased, while at the same time significantly reducing the scale of experiments. In addition, additional variation throughout the entire length of the HBV genome is introduced when error-prone T7 polymerase is used to transcribe the plasmid DNA library.

The plasmid DNA libraries are generated by following the protocol described by Wrenbeck et al. (Wrenbeck et al., Nature Methods 13, 928-930 (2016)). As an example, five sub libraries are generated to cover the entire HBV genome. Within each segment of the HBV genome, individual codons are randomized (NNN) such that each plasmid molecule harbors one randomized codon, which is referred to as the “primary library.” Next, this primary library is used as a template, and the process is repeated to generate a “secondary library” where each plasmid molecule will have two randomized codons that include every possible codon pairing.

As a proof-of-concept for this approach, whether LAM-resistance mutations from a population of in vitro-transcribed WT pgRNA can be enriched was tested. Two concentrations of LAM based on the inhibition curves were chosen. Two days after transfecting Huh-7.5-NTCP cells with pgRNA, virus-containing cell supernatants were harvested to isolate viral DNA. Viral DNA was then amplified by PCR (FIG. 5B) and sequenced by MiSeq. Remarkably, it was found that with only a single round of selection, several known Lamivudine-resistance mutations were enriched in the population. The statistical analysis of the mutations carried by the HBV viral variants is shown in FIG. 5C, which shows M204V and M204I are the most frequently identified mutations carried by the HBV viral variants screened against LAM. This demonstrates that the RNA launch method can be used to identify mutations that confer drug resistance.

Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates which may need to be independently confirmed.

All of the methods and apparatus disclosed and claimed herein can be made and executed without undue experimentation in light of the present disclosure. While the invention has been described in terms of preferred embodiments, it will be apparent to those having ordinary skill in the art that variations may be applied to the apparatus, methods, and sequence of steps of the method without departing from the concept, spirit, and scope of the invention. More specifically, it will be apparent that certain components may be added to, combined with, or substituted for the components described herein while the same or similar results would be achieved. All such similar substitutes and modifications apparent to those having ordinary skill in the art are deemed to be within the spirit, scope, and concept of the invention as defined.

1. A method of introducing HBV genome in a cell, comprising:
contacting a cell with a nucleic acid molecule comprising an HBV pregenomic RNA from an HBV genotype; and
culturing the cell under a condition to initiate reverse transcription from the HBV pregenomic RNA and HBV replication.

2. The method of claim 1, further comprising:
determining a level of HBV infection by quantifying the amount of one or more HBV proteins or HBV nucleic acids.

3. A method for screening an HBV modulator for the ability to increase or decrease HBV infection, comprising:
providing a cell having a nucleic acid molecule comprising an HBV pregenomic RNA from an HBV genotype;
determining an amount of one or more HBV proteins or HBV nucleic acids in the presence of an HBV modulator; and
determining whether the HBV modulator increases or decreases HBV infection by comparing the amount to a reference amount that is obtained in the same manner except in the absence of the HBV modulator, whereby:
the HBV modulator increases HBV infection if the amount is higher than the reference amount, and
the HBV modulator decreases HBV infection if the amount is lower than the reference amount.

4. The method of claim 1, wherein the one or more HBV proteins comprise one or more HBV surface antigens.

5. The method of claim 4, wherein the one or more HBV surface antigens comprise HBsAg.

6. The method of claim 4, wherein quantification of the amount of the one or more HBV proteins is performed by CLIA.

7. The method of claim 3, wherein quantification of the amount of the one or more HBV nucleic acids is performed by qPCR.

8. A method of identifying an HBV viral variant with resistance or decreased sensitivity to an HBV modulator, comprising:
(a) contacting a cell with a nucleic acid molecule comprising an HBV pregenomic RNA from an HBV genotype;
(b) contacting the cell with an HBV modulator;
(c) culturing the cell to form a cell culture under a condition to initiate reverse transcription from the HBV pregenomic RNA and HBV replication;
(d) obtaining an HBV DNA from the cell or a supernatant in the cell culture; and
(e) determining a mutation in the nucleotide sequence of the HBV DNA by sequencing the obtained HBV DNA, whereby an HBV viral variant having the mutation is identified as the HBV viral variant with resistance or decreased sensitivity to the HBV modulator.

9. The method of claim 8, further comprising:
(f) obtaining a second nucleic acid molecule comprising an HBV pregenomic RNA by in vitro transcription from the obtained HBV DNA; and
(g) repeating steps (a) to (d), (f), and optionally step (e) one or more times.

10. The method of claim 8, wherein sequencing the obtained HBV DNA comprises is carried out by next-generation sequencing (NGS).

11. The method of claim 3, wherein the HBV modulator is an anti-HBV modulator, having the ability to reduce HBV infection.

12. The method of claim 3, wherein the HBV modulator is a small molecule, an antibody, an aptamer, or an inhibitory nucleic acid molecule.

13. The method of claim 8, wherein the step of culturing the cell is performed in the presence of the HBV modulator.

14. A method of introducing HBV infection in an animal model, comprising:
introducing into the liver of an animal model a nucleic acid molecule comprising an HBV pregenomic RNA from an HBV genotype.

15. The method of claim 14, wherein the animal model is a human liver chimeric mouse.

16. The method of claim 15, wherein the human liver chimeric mouse has a high degree of human hepatocyte engraftment.

17. The method of claim 14, wherein the step of introducing further comprising introducing into a larger liver lobe of the animal model the nucleic acid molecule comprising an HBV pregenomic RNA from an HBV genotype.

18. The method of claim 1, wherein the nucleic acid molecule is in vitro transcribed from an HBV DNA of the HBV genotype.

19. A kit for introducing HBV genome in a cell, comprising:
(a) a nucleic acid molecule comprising an HBV pregenomic RNA from an HBV genotype;
(b) a DNA molecule having a binding site for an RNA polymerase and a DNA sequence encoding an HBV pregenomic RNA from an HBV genotype; and optionally the RNA polymerase, one or more nucleoside triphosphates, and a buffer; or
(c) a plurality of DNA molecules generated by randomizing individual codons, each of the plurality of DNA molecules having a binding site for an RNA polymerase and a DNA sequence encoding an HBV pregenomic RNA from an HBV genotype; and optionally the RNA polymerase, one or more nucleoside triphosphates, and a buffer.

20. (canceled)

21. (canceled)

22. The method of claim 1, wherein the HBV genotype is selected from the group consisting of HBV genotypes A, B, C, D, E, F, G, H, I, and J.

23. The method of claim 1, wherein the nucleic acid molecule is polyadenylated.

24. The method of claim 1, wherein the nucleic acid molecule is a 5′-capped nucleic acid molecule.

25. The method of claim 1, wherein the cell is a human cell or a higher primate cell.

26. The method of claim 1, wherein the cell is a HepG2 cell, a HepG2-NTCP cell, a Huh 7 cell, a Huh-7.5 cell, a Huh7.5-NTCP cell, a HepRG cell, a 293T cell, a HLC cell, or a human primary hepatocyte (PHH).
